(19) United States
(12) Patent Application Publication
(10) Pub. No.: US 2024/0335550 A1
(43) Pub. Date: Oct. 10, 2024

(54) MUCOSAL FORM FOR DRUG DELIVERY THROUGH THE MUCOSA AND METHODS OF TREATMENT WITH SAME

(71) Applicant: Sleep Solutions of Texas, LLC, Tyler, TX (US)

(72) Inventor: Raghavendra Vitthalrao GHUGE, Tyler, TX (US)

(73) Assignee: Sleep Solutions of Texas, LLC, Tyler, TX (US)

(21) Appl. No.: 18/628,261

(22) Filed: Apr. 5, 2024

Related U.S. Application Data
(60) Provisional application No. 63/494,794, filed on Apr. 7, 2023.

(51) Int. Cl. A61K 47/64; A61K 9/00; A61K 9/70; A61K 31/165; A61K 31/513; A61K 31/573; A61K 31/58; A61K 41/00

(52) U.S. Cl. CPC A61K 47/6435; A61K 9/006; A61K 9/7007; A61K 31/165; A61K 31/513; A61K 31/573; A61K 31/58; A61K 41/0042

(57) ABSTRACT

Mucosal patches are disclosed that have a support layer supporting an active layer that has a collagen carbon dot nanocomposite carrying an active agent. The collagen carbon dot nanocomposite is absorbable through a mucosa. The mucosal patches are part of various methods of treatment.

### RELATED APPLICATIONS

This application claims the benefit of U.S. Provisional Application No. 63/494,794, filed Apr. 7, 2023, which is herein incorporated by reference in its entirety.

### TECHNICAL FIELD

This application relates to mucosal patches, more particularly, to a mucosal patch that has a support layer and an active layer thereon, the active layer has a collagen carbon dot nanocomposite that is carrying an active agent that is absorbable through mucosa.

### BACKGROUND

Mucosal absorption of medications has been traditionally performed through tongue mucosa, vaginal mucosa, and mucosa of the GI tract. Drugs absorbed from GI tract undergo first pass metabolism in the liver, which decreases the dosage successfully delivered to a target cite and requires a user to intake a higher dosage in the first instance. Although these routes have their importance in delivering medications for many medical problems, they are particularly poor at delivery of medications to the central nervous system. The central nervous system is unique because the blood brain barrier causes difficulty with absorption of medications through the arterial tree to the brain. As such, larger amounts of medication must be administered for even a small amount to be absorbed by the brain. Even medications absorbed through the tongue mucosa have irregular absorption mechanics and delivery to the central nervous system, rendering absorption of medications unpredictable. Systemic side effects can occur in the user because of the larger amount of medication required to reach the brain.

Drugs absorbed through the GI tract must undergo first pass metabolism in the liver and this can be affected by liver disease. It can increase blood levels of toxic metabolites and/or result in organ damage. These negative side effects restrict the use of medications that have toxic metabolites although the medication itself might have been useful.

Some drugs work better at a specific time of the day or night. If such a time is inconvenient for the patient, they may skip the dose. One example is sodium oxybate, which is to be taken at 1 or 2 AM (second dose). Many patients skip this does. Another example is sublingual Zolpidem that needs to be taken at 2 AM to avoid the 3 or 4 AM awakening. By the time the patient wakes up at 3 or 4 AM, they are unable to fall back to sleep. It is too late to take the drug due to lingering drowsiness at 6 or 7 AM. So, the drug company had to state that it should not be taken after 3 AM to prevent drowsiness while driving. There is an unmet need to be able to take the drug at bedtime to be effective at 2 AM.

Drug overdose and poison ingestion require rapid absorption of antidote. Normally antidotes are given orally, but with the unpredictable absorption discussed above and the risk of nausea and vomiting, better delivery vehicles are needed.

Some drugs, especially those for treating nausea and vomiting which can occur during cancer and chemotherapy treatments and pregnancy, for example, may be hard to keep down. As such, the drug delivery vehicle often ends up being rectal suppository, which are highly inconvenient.

There is a need for a better drug delivery vehicle that addresses and overcomes the difficulties discussed above.

### SUMMARY

Disclosed herein is a mucosal patch for absorption of an active agent through any mucosal membrane for direct delivery to the brain and/or circulation where targeted.

In one aspect, mucosal patches have a support layer supporting an active layer. The active layer includes a collagen carbon dot nanocomposite carrying an active agent. The collagen carbon dot nanocomposite is absorbable through a mucosa, such as palate, tongue, cheek, gums, nasal, skin, eyes, ears mucosa. The support layer can include a mixture of polytetrafluoroethylene (PTFE) selected from the group consisting of expanded-PTFE (e-PTFE), dense-PTFE (d-PTFE), and highly dense-PTFE (n-PTFE). In one embodiment, the ratio of e-PTFE to d-PTFE is in a range of 4:1 to 1:4.

In some embodiment, the support layer is dissolvable or bioabsorbable. This support layer can be made from any one or more of cellulose, gelatin, and collagen.

In some embodiment, the mucosal patch has a protective layer in direct contact with a mucosa-contact surface of the active layer. This protective layer is dissolvable or bioabsorbable or is removable. In one embodiment, the protective layer is dissolvable, which may be triggered by moisture or heat. The mucosal patch can have an adhesive outermost portion defining a perimeter of the active layer or the active layer can include a bioadhesive configured for adhesion to a mucosa.

In some embodiment, the mucosal patch can include an intermediate layer between the support layer and the active layer.

In another aspect, methods of treatment are disclosed that include any one of the mucosal patches described herein. The method includes providing such a mucosal patch, contacting a mucosa of a user with the active layer of the mucosal patch for a period of time sufficient to deliver the collagen carbon dot nanocomposite to a target site in need of treatment, and, subsequent to contacting the mucosa, exposing the target site to a wavelength of radiation that cleaves the active from the collagen carbon dot nanocomposite. The method can include, before exposing the target site, scanning the target site to determine if a treatment concentration of the active agent is present at the target site.

In one embodiment, the mucosa is the skin, and the active agent is selected from the group consisting of encapsulate insulin, vitamin E, vitamin A, vitamin C, vitamin B complex, vitamin D3, and combinations thereof. Here, exposing the target site to a wavelength of radiation includes a wavelength of radiation to cleave the collagen from the carbon dot.

In another embodiment, the treatment is for androgenetic alopecia, the mucosa is in the oral cavity, and the active agent comprises finasteride and/or dutasteride. The active can also include minoxidil, flutamide, and/or prednisone.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present system.

FIG. 1 is a longitudinal cross-sectional view showing the layers of a first embodiment of a mucosal patch.

FIG. 2 is a longitudinal cross-section view showing layers of a second embodiment of a mucosal patch.

FIG. 3 is a chart of active agent and their respective dosage if delivered via the tongue, palate, or skin.

FIG. 4 is a side perspective view of a third embodiment of a mucosal patch connected to a mandibular teeth covering.

### DETAILED DESCRIPTION

The following detailed description will illustrate the general principles of the invention, examples of which are additionally illustrated in the accompanying drawings. In the drawings, like reference numbers indicate identical or functionally similar elements.

As used herein, patch means a generally small piece of material that embodies single or multiple layers made out of or containing chemicals (solid, liquid, gaseous) or biological materials, active and inactive ingredients, supportive structure, fixed or mobile, rigid or flexible, shaped in whichever way necessary to create contact with the selected mucosa. When the patch is intended for delivery in the oral cavity for placement against any of the mucosa therein, it can be referred to as a wafer.

As used herein, carbon dots are a class of fluorescent small-carbon nanomaterials with particle sizes of less than 10 nm. They and have wide ranging applications in the fields of bioimaging, drug delivery, biosensing, disease detection, synthetic chemistry, and materials science. These nanomaterials are water soluble with low toxicities and low production costs, offer tunable fluorescence emission and excitation, are photochemically and physiochemically stable, and have high biocompatibility. Carbon dots were discovered in 2004 during the purification of single wall carbon nanotubes (SWCNTs) via preparative electrophoresis. Applicant's use of carbon dots throughout this application is understood as being an equivalent of any type of carbon nanotube or other allotrope of carbon and can be replaced thereby in all applications, such as with SWCNTs or muti-walled carbon nanotubes (MWCNTs).

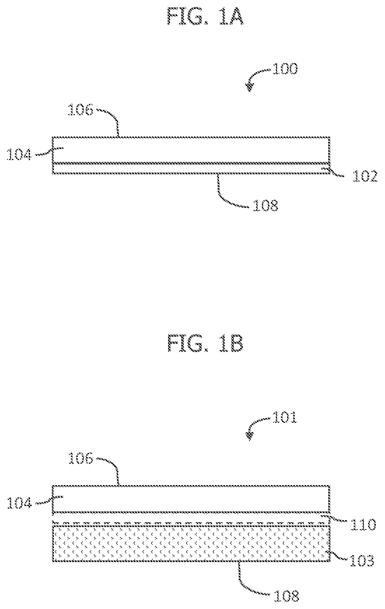

Referring now to a first embodiment shown in FIG. 1A, a mucosal patch 100 has a support layer 102 supporting an active layer 104. The active layer 104 defines a mucosal contact surface 106 and the support layer defines an outermost surface 108 opposite the mucosal contact surface. In this embodiment, the support layer 102 is soluble (dissolvable) or bioabsorbable. The support layer 102 is preferably flexible, having enough flexibility to conform to a user's palatal surface, skin, nasal cavity, or other mucosal tissues. The active layer 104 includes a collagen carbon dot nanocomposite that is carrying an active agent, the collagen carbon dot nanocomposite being one that is absorbable through a mucosa. Herein any reference to the active agent as being absorbable is meant to include the collagen carbon dot nanocomposite and vice versa because it is this nanocomposite that is tagged with said active agent. The active layer 104 of the mucosal patch 100 may interact with the selected mucosa to open inter-cellular pores, open intracellular or trans-cellular absorption mechanisms, or generate carrier-facilitated transfer of chemicals or biological materials across the mucosa or into the blood stream.

In the first embodiment, the support layer 102 has a composition that will dissolve in a time period (T) that is greater than the time period set for the active layer 104 to absorb the active agent/nanocomposite into the mucosa. In some embodiments, the support layer should remain for an additional 15% of the time beyond the time required for the collagen carbon dot nanocomposite to be absorbed in addition to the time the support layer itself requires to dissolve (i.e., T1 or T2), but is not limited thereto. If the support layer is an immediate release layer, the time it takes to dissolve is defined as T1, and if the support layer is a delayed release layer, the time it takes to dissolve is defined as T2, wherein T2 is always greater than T1. The additional 15% of time for the support layer to completely dissolve allows enough time for biological factors that may delay absorption of the drug.

Referring now to a second embodiment shown in FIG. 1B, a mucosal patch 101 has a support layer 103 supporting an active layer 104, and an optional intermediate layer 110. The active layer 104 defines a mucosal contact surface 106 and the support layer defines an outermost surface 108 opposite the mucosal contact surface. In this embodiment, the support layer 103 is insoluble (non-dissolvable) but is preferably permeable to saliva when intended for use in the oral cavity while being impermeable to the CL/CD nanocomposite. The permeability is such that saliva and/or salivary enzymes from the tongue and/or oral cavity are absorbed into the active layer 104 after passing through the pores in the permeable insoluble support layer 103. The support layer 103 is preferably flexible, having enough flexibility to conform to a user's palatal surface, skin, nasal cavity, or other mucosal tissues. The active layer 104 includes a collagen carbon dot nanocomposite that is carrying an active agent, the collagen carbon dot nanocomposite being one that is absorbable through a mucosa.

Here, the intermediate layer 110 is one that is dissolvable or thermally soluble and its presence provides a delay to the activation of the active agent in the active layer. Intermediate layer 110 may be the same composition as that of the support layer 102 in FIG. 1A.

We can deliver the active agent in the form of a mucosal patch onto and through various mucosal membranes, such as those of the palate, tongue, cheeks, gums, nasal passage, skin, eyes, and ears. With respect to the ears, the external auditory canal has a transition between skin and mucosa as it approaches the ear drum. Also, the active agent may be absorbed through the ear drum (tympanic membrane) into the inner ear.

In all embodiments, the support layer 102, 103 as noted above can be soluble (dissolvable) or insoluble (non-dissolvable) and is simply meant to hold the active agent in place, i.e., to not allow the active agent to leak or release through the support layer. The active agent is not intended to be swallowed. For soluble embodiments, the support layer can be a homogenous wafer-thin surface made form material such as cellulose, gelatin, biodegradable polymers, or equivalents thereof, or a matrix of collagen fibers (slowly dissolvable, i.e., dissolving slower than the active layer 104) using CL1-CL7 types of collagens individually or mixtures thereof. When the support layer is soluble, it comprises materials combined to provide sufficient delay in its absorption such that the active layer 104 undergoes absorption before the support layer 102.

When the support layer 102 is insoluble, it may comprise a material that is reusable, in particular a material that can be sanitized and reused. In embodiments that have an insoluble support layer, the support layer can be a layer of or including polytetrafluoroethylene (PTFE), also known as TEFLON™. The PTFE may be a matrix variable density PTFE. The matrix variable density PTFE will contain variable customizable ratios of e-PTFE, d-PTFE, n-PTFE. E-PTFE has larger pores, d-PTFE is dense and has sub-micron pores, and n-PTFE is highly dense. d-PTFE is used in bone and tissue regeneration, and n-PTFE is used in grafts. The matrix variable density PTFE can have e-PTFE to d-PTFE in a ratio in a range of 4:1 to 1:4. The support lay may be plastic, heavy coated paper, nylon, rayon, cotton, or silk.

In all embodiment, the support layer 102 can include taste masking agents or taste masking properties so as to mask any uncomfortable or undesirable taste associated with the active agent.

Turning now to the active layer 104, the active layer includes an active agent that may turn active upon contact with a selected mucosa, turn active upon exposure to temperature, moisture, electrical or electromagnetic frequency impulse, photonic energy, radioactive energy, ultraviolet radiation, or can be activated when the carbon portion of the nanocomposite is activated. The active layer 104 comprises a collagen carbon dot nanocomposite that is carrying the active agent. An example for making such a nanocomposite is set forth below in the working examples. The collagen is at least one of the fibrillar types I/II/III/IV/V/VI/VII and combinations thereof. The nanocomposite formation yields a collagen/carbon dot (CL/CD) nanocomposite, for example CL1/CD, CL2/CD, CL3CD, CL4/CD, CL5/CD, CL6/CD, CL7/CD and combinations thereof. The CL/CD nanocomposite can be used directly for release of the collagen molecule to a target site and may be an immediate release composition or a delayed release composition. Activation of the CL/CD nanocomposites occurs at various separate frequencies, such as 210 nm, 260 nm, 310 nm, 400 nm, and 510 nm), light emitting diodes (LED) that emit one or more of these frequencies can be used to activate the release of collagen. An aluminum nitride LED emits at 210 nm, which is deep ultraviolet (UV) radiation. Excimer Lasers and mercury lamps provide deep UV spectrum (200-300 nm), and gallium nitride Lasers provide near-UV spectrum (300-400 nm). Any of these radiation sources are suitable and will be selected as needed for activation of a selected active agent for a selected target site, such as tissue or organ and location dependent.

The active layer 104 may comprise a plurality of sequential sublayers sequenced for sequential or simultaneous absorption. If sequential, sublayers will be stacked one on top of another. The sublayers may be directly contacting coatings of the active agent or alternating sublayers containing active agent with the intermediate sublayer(s) bearing no active agent to function as a time delay for subsequent absorption of the underlying sublayer. If simultaneous, coatings forming the sublayer are juxtaposed to one another and can contain different active agents. In other embodiments, a single active layer can comprise a mixture of CL/CD nanocomposites having different active agents.

The active layer 104 can be applied to the substrate by a stamping method, a painting method, a spraying method, a coating method, or other known or hereinafter developed commercially acceptable methods. In one embodiment, the active agent is impregnated into the active layer 104. In one embodiment, a 1 μm to 10 μm thick layer of PTFE (Matrix Variable Density) shaped and sized to fit the palatine mucosa or as a skin patch (example being 10 mm by 10 mm) is cut using assembly line processes. The patch is stretched and secured. Next, a fixed amount of CL/CD composite with attached active ingredient was painted on each patch, the fixed amount being the dose of the medication that is to be released. Thereafter, the patch is dried, and adhesive material is added, if desired. I needed, the adhesive is dried, and then the patches are packaged for distribution and sale. Other embodiments will need multiple stages of stamping or painting to create multi-layer patch. The active agent may be present in the active layer 104 as a solid, liquid, or semi-solid.

The active layer 104 when in contact with the mucosa will enable the active agent to absorb into the mucosa. When the mucosa is in the oral cavity, such as the palate, gums, cheeks, tongue, salivary enzymes activate absorption of the active agent. In other embodiments, application of heat, whether body temperature or external, activates absorption of the active agent. In some embodiments, the active layer 104 can include an additive to increase the rate of absorption through the mucosa. In one embodiment, the additive creates vasodilation of palatal mucosal blood vessels to enhance absorption of active chemical. These may include properties configured towards opening up the clefts in the stratified squamous epithelium of the palatal mucosa, widening inter-cellular gaps, activating cell surface channels of absorption such as activation of cyclic-AMP, cyclic-GMP, Na-K-ATPase pump and other channels specific to each active chemical that is configured for a unique end-user thus facilitating an active or passive uptake of the active agent. These sub-cellular chemical activation process could be sequenced to create differential stages of absorption of single or multiple active agents by making use of single or multiple qualities of passive transport (simple diffusion, carrier-mediated transport, or channel-mediated transport) or active transport using energy-dependent (ATP consumption) processes. Some examples include, but are not limited to, adenylyl cyclase, an enzymes that activates cyclic AMP and activates protein Kinase A (PKA) which is responsible for most of the effects of cAMP within the cell, and guanylyl cyclase (GC or guanylate cyclase), which catalyzes cGMP converting CTP to cGMP. The activation of cGMP or guanylyl cyclase (which increases levels of cGMP) causes relaxation of vascular smooth muscle thus causing the absorption of medications and chemicals through the wall of micro-vessels like capillaries. Insulin can activate Na-K-ATPase pump. Insulin molecules can be used to open trans-cellular uptake of drugs tagged with glucose.

### Active Agent

In some embodiments, the active agent is meant for therapeutics.

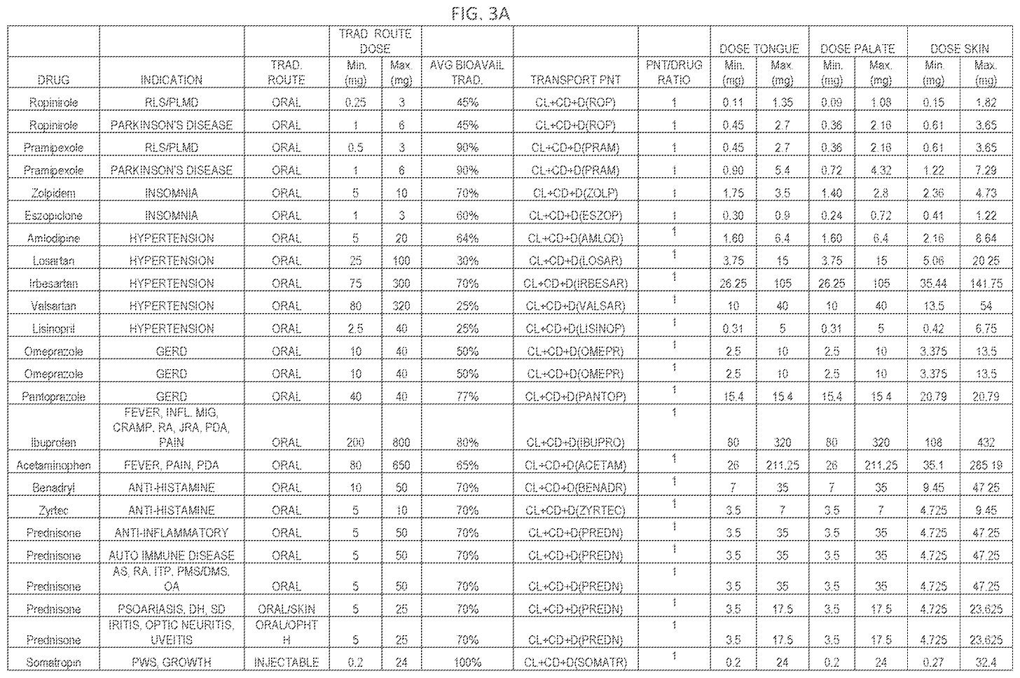

Example active agents include, but are not limited to, sodium oxybate, ropinirole, pramipexole, zolpidem, eszopiclone, zaleplon, benzodiazepines, nanosheets of doped nanodots that can provide diagnostic and therapeutic effects, amlodipine, clonidine, modafinil, stimulants, nausea reducing agents, human growth hormone, thyroid supplements, vitamins, antivenoms, antidotes for poisons, antidotes for overdoses, and combinations thereof. Example vitamins include, but are not limited to, Vitamin B12, A, C, D, and E. Also see FIG. 3.

These active agents and the mucosal patches disclosed herein can be used to treat narcolepsy, obstructive sleep apnea, RLS, PLMD, insomnia, bruxism, cancer, pain associated with illnesses, such as cancer, poisonings, venomous bites, overdoses, vomiting, nausea, drowsiness, jet lag, circadian rhythm abnormalities, shift-workers sleep disorder, attention deficit disorder, ADHA, hair loss. The active agents may also administer chemotherapy.

In other embodiments, the active agent is meant for theragnostics. Theragnostics is a combination of diagnostic and therapeutic modalities. An example is combining a radioactive drug that diagnoses and a second radioactive drug that treats cancer. The role of using nanotechnology to deliver the active drug to the target organ or cell to diagnose the presence of medical conditions such as lymphoma, leukemia, thyroid cancer, and also deliver an active drug to the same organ or cell to destroy the organ or cell. This is valuable in that it prevents loss of medication to hepatic first pass metabolism or to other organs where they may cause damage

In yet other embodiments, the active agent is meant for diagnostics. For example, the patch can include an active agent that can be used for brain imaging. One example is 2-deoxy glucose, which can be directly transported to the brain. This active agent is useful for diagnosing the amount of brain activity inside neurons or group of neurons to demonstrate viable brain function or pick up brain or meningeal cancer.

In all embodiments, collagen carbon dot nanocomposites are “tagged” with active agents that may be activated upon coming in contact with certain intercellular enzyme or receptor systems of the mucosal lining or certain active enzymes and receptors on the cell surface that actively or passively absorb and transport the CL/CD nanocomposite into the mucosal cell and once inside are activated such that the active agent is released therefrom. The CL/CD nanocomposite “tagging” can specifically target an active agent to a specific organ or cell group and may assist in initiating the onset of action or controlling the reaction of the active agent at a cellular level, DNA or RNA level. The CL/CD nanocomposite tagging may activate the DNA, RNA, or cellular metabolism of function directly to prime them or to create a favorable condition for action of the active agent and may improve the cellular function as in medical conditions that are treated by the active agent or replenish deficient chemical(s) in the cell or in the body. In some embodiments, the CL/CD nanocomposite is tagged with an active agent that can damage the cell or its structure as in case of a cancer treatment.

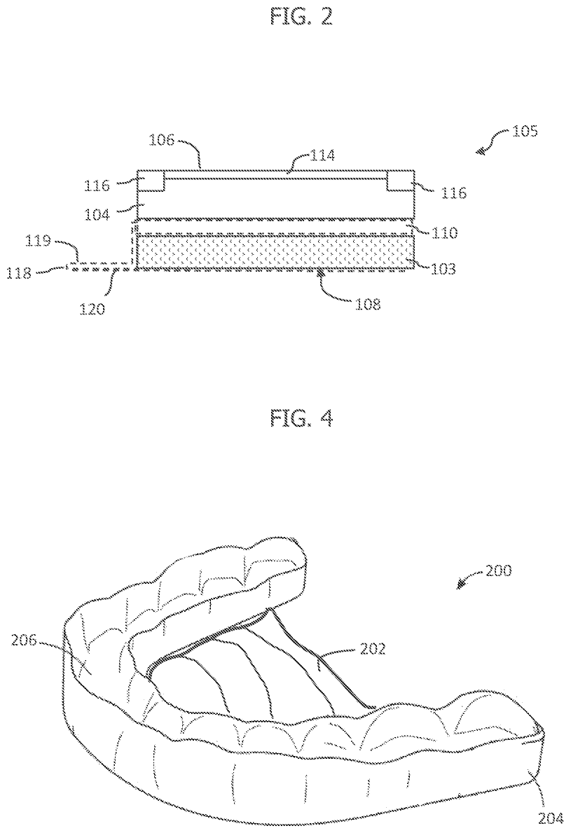

Turning now to FIG. 2, in another embodiment, the mucosal patch 105 has a support layer 103 supporting an active layer 104, and an optional intermediate layer 110 therebetween, as described above for FIG. 1B. In addition, the mucosal patch 105 can include a protective layer 114 in direct contact with the outer surface of active layer 104, the protective layer 114 thereby defining the mucosal contact layer 106. This protective layer 114 is a soluble (dissolvable) layer. In one embodiment, the protective layer 114 is thermally activated and once solubilized or dissolved exposes the active layer 104 to the mucosa for absorption of the active agent. In another embodiment, the protective layer 114 is moisture activated and once solubilized or dissolved exposes the active layer 104 to the mucosa for absorption of the active agent. In yet another embodiment, the protective layer 114 is activatable by both moisture and heat.

The protective layer 114 will dissolve or solubilize in a time (T4) for immediate release and for a time (T5) for delayed release, wherein T5 is always greater than T4. The thickness and composition formulation of the protective layer can be adjusted to provide a preselected time delay for the onset of absorption. The time delay can be as little as 5 to 10 minutes up to multiple hours. One example for a multiple hour time delay is the administration of a dose of sodium oxybate or Zolpidem to the palatal mucosa for a patient's nighttime administration thereof while the patient is asleep. The mucosal patch is placed in the oral cavity at bedtime and is released later at a prescribed time to be actively absorbed and available in the brain.

A portion of the mucosal contact surface 106 can include a biocompatible adhesive 116. In one embodiment, the adhesive 116 forms all or part of the perimeter of the mucosal contact surface 106. In another embodiment, the adhesive 116 is present as discrete defined surface areas of the mucosal surface 106, such as geometrically shaped spaced apart spots of adhesive material along any dimension of the patch. Any commercially available or herein after developed biocompatible adhesive is suitable.

Still referring to FIG. 2, the mucosal patch 105 can include an extraction strip extending from the support layer 103. The extraction strip 103 extends from the patch in a manner suitable for being grasped by a hand of a user for application of the mucosal patch to the desired mucosa. If the mucosa is the palate, the extraction strip 118 is such that a user can grasp the same and insert the patch into the user's mouth with the extraction strip extending anteriorly to the exterior of the oral cavity. The extraction strip can rest between the user's teeth with absorption occurs. Once absorption is complete, the user can extract the non-dissolvable support layer 103 via the extraction strip 118.

In one embodiment, the extraction strip includes an indicia of an amount of absorption or an elapsing of a time period equivalent to an amount of absorption as it occurs on an upper surface 119 thereof. For example, the upper surface may have a series of + signs or boxes with check marks or words as the indicia that appear as absorption occurs/time passes. In one embodiment, a first plus sign appears as absorption begins, a second plus sign appears at 50% absorption, and a third plus sign appears at a 100% absorption, thereby indicating when to remove the non-dissolvable portion of the patch from the mucosa. In another embodiment, the indicator will be a color changing indicator that changes with time. The color change will indicate the completion of absorption, so that the user will know when to extract or remove the non-absorbable support layer from the mucosa.

Still referring to FIG. 2, optionally, a removable or nonremovable micro-film 120 can be present on the outermost surface 108 of the support layer 102, 103. When the substrate is a dissolvable substrate, the micro-film will be removable (peeled away before use of the mucosal patch). In those embodiments having a non-dissolvable substrate 103, the micro-film 120 can be removable, functioning only a protective layer prior to use. In one embodiment, the micro-film 120 is impermeable to saliva and is attached to the external extraction strip 118. Upon completion of the entire process, a user will extract the micro-film using the extraction strip 118 and any other non-absorbable components. In another embodiment only the micro-film 120 is non-absorbable, and once removed from the user, saliva will cause all bio-degradable polymer(s) to dissolve, be swallowed and absorbed in the GI tract.

In all embodiments, the mucosal patches can be disposable and can be for immediate release or delayed release of the active agent. In all embodiments, the layered up or stacked construction of the mucosal patches disclosed herein are very thin and flexible and will be individually packaged with precise dosage amounts of the active agent for single individual use. The mucosal patch can be made in any shape (e.g., circle, oval, square, etc.) according to the design created by anyone who is familiar with the art and manufacturing of pharmaceutical agents for human or animal use.

A user would simply open the packet, remove the mucosal patch, place it on the desired mucosa. In one embodiment, the mucosal patch is configured to be placed on the dorsal surface of the tongue and the user then places their tongue against the palatal mucosa, thereby pressing the mucosal patch's mucosal contact surface against the palatal mucosa. In the embodiments of FIG. 1A to FIG. 2, the mucosal patches are each configured to adhere to the palatal mucosal surface upon contact, either through a biocompatible adhesive or the composition of the layer defining the mucosal contact surface 106 without additional elements to attach it to the palatal mucosa.

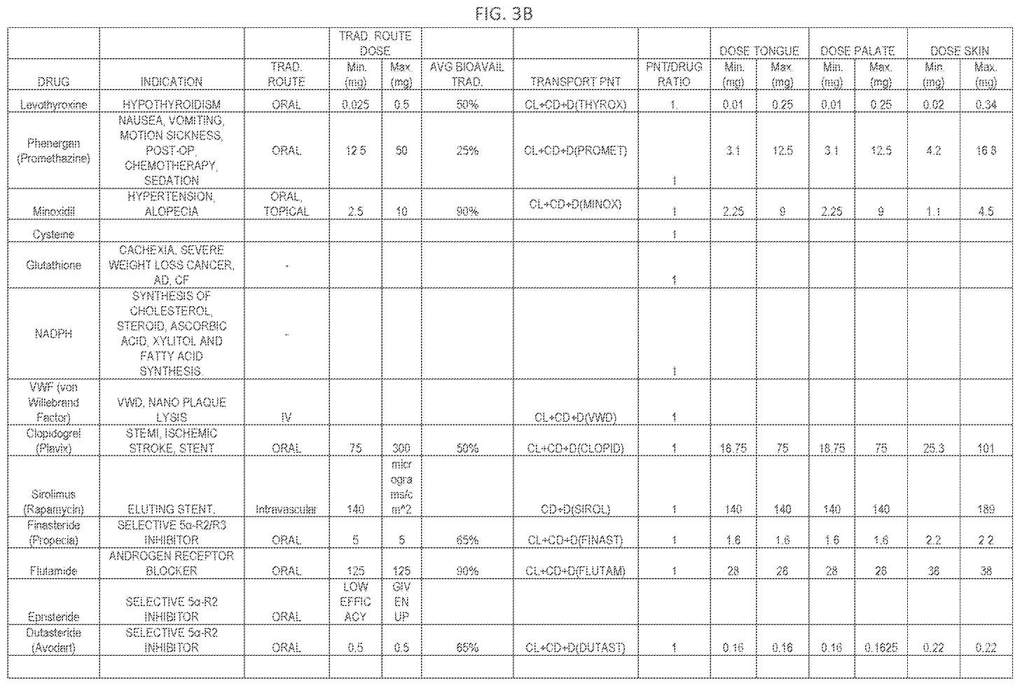

In the embodiment, of FIG. 4, a mandibular mouthpiece 200 is shown that has a palatal patch 202 that can be connected to or integral with a mandibular teeth covering 204 that defines a channel 206.

In all embodiments, the dosage of the active agent is configured for the patch size and absorption rate of the active agent relative to the desired effect at the target sight. The mucosal patches will be available in different dosage amounts for most or all active agents. The same example dosages are presented in FIG. 3.

### Working Examples

Creation of Specific Drug Encapsulation into CL/CD Nanocomposite

Collagen powder (of at least one of the Fibrillar Types I/II/III/IV/V/VI/VII) is used to produce carbon dots using the following process. A gram of collagen powder is subjected to heating in a muffle furnace at 300° C. for 2 hours. On cooling, the powder is collected and dispersed in water and centrifuged to obtain carbon dots produced by the carbonization of the collagen. Next, a preselected amount of collagen is dissolved in 2% acetic acid (98 ml water and 2 ml of acetic acid). 5 wt % of the collagen carbon dot collected from the muffle furnace are added to the mixture of collagen and acetic acid and this mixture is maintained at 80° C. for 30 min. A precipitate is collected after heating and is washed 3-4 times until all remnants of the acetic acid have been removed. The precipitate is dried in an air oven at 60° C. or less until fully dried. This process yields a collagen/carbon dot (CL/CD) nanocomposite, for example CL1/CD, CL2/CD, CL3CD, CL4/CD, CL5/CD, CL6/CD, or CL7/CD. All references to carbon dots herein include any type of carbon nanotubes.

Next, 0.02 g of collagen carbon dot nanocomposite is dissolved in optimal amount of water to form a solution. With continuous stirring for two hours, thiamine pyrophosphate (TPP) and Tween80 are added to the solution. Then 0.2 g of active drug (Ropinirole, Dopamine, rHGH, etc.) is added with continuous stirring and sonication for one hour. This provides a 1:1 ratio of CL/CD to active agent for a 1:1 encapsulation. The mixture is allowed to stand overnight for formation of a precipitate of the encapsulation product of CL/CD+ active agent. Thereafter, the precipitate was collected and dried. Herein, this product is referred to as a collagen carbon dot active agent nanocomposite or a collagen carbon dot nanocomposite comprising an active agent. In FIG. 3, this is referred to by shorthand as: CL+CD+D (collagen+carbon dot+drug).

The active agent (or “drug”) can be any such that is typically delivered through the GI tract and has molecular weight less than 1000 Daltons (Da). Mucosal membranes allow passage of molecules below 1000 Da with ease. The molecular weight of the collagen carbon dot active agent nanocomposite is that of the active agent plus the collagen carbon dot. For example, the molecular weight (MW) of rHGH (recombinant Human Growth Hormone) is 848.9 Daltons and the MW of CL/CD is 3 Daltons; thus, the encapsulated MW of rHGH+CL/CD is 851.9 Da. We label the encapsulated MW as MW+. For example, the MW of Ropinirole is 296.83 Da and its MW+ is 299.83 Da.

### Use of a CL/CD Nanocomposite for Treatment of Hair Loss

Delivery of an active agent to the dermal papilla is discussed below that accelerates hair production and growth in a person with androgenetic alopecia. The delivery is through a mucosal patch impregnated with liquified, dispersed, or consolidated dose(s) of a collagen carbon dot nanocomposite carrying an active agent, referred to as a drug nanoparticle. Example active agents for said drug nanoparticles with dose amounts useful for treating androgenetic alopecia are presented in TABLE 1 below.

TABLE 1

| | | CDT | P/T | | | Total |
|---|---|---|---|---|---|---|
| Active | | tagged | transport | PNT/D | Patch | PNT/D |
| Agent | Action | (Da) | dose (mg) | dose (mg) | dose (mg) | dose (mg) |
| finasteride | 5AR2 enzyme inhibitor | 375.5 | 1.6 | 3.5 | 2.2 | 4.4 |
| prednisone | Upregulate AROM production | 361.4 | 3.5 | 7.0 | 4.7 | 9.5 |
| minoxidil | Upregulate STP synthase through Ca²⁺ influx | 212.2 | 2.2 | 4.5 | 1.1 | 2.2 |
| dutasteride | selective 5AR1/5AR2 inhibitor | 531.4 | 0.16 | 0.32 | 0.22 | 0.44 |
| flutamide | Androgen Receptor blocker | 279.2 | 28.1 | 56.2 | 37.9 | 75.9 |

*5AR1 = 5-Alpha Reductase 1-Inhibitor;
5AR2 = 5-Alpha Reductase 2-Inhibitor

Minoxidil also promotes stem cell differentiation and blood flow to the dermal papilla. The mucosal patch can include one of the above active agents or a combination of two or more of the above active agents. If the mucosal patch has a single active agent, the user can take a plurality thereof in sequence to administer a combination of the above active agents in a pre-selected sequence. In one embodiment, a mucosal patch includes a combination of drug nanoparticles carrying finasteride, prednisone, minoxidil, and flutamide. In another embodiment, a mucosal patch includes a combination of drug nanoparticles carrying dutasteride, and flutamide. In yet another embodiment, the mucosal patch includes a combination of finasteride, prednisone, minoxidil, dutasteride, and flutamide according to Table 1 above. Here, the active agents can be present as discrete sublayers of the active drug layer such that the active agents are administered via absorption into the palatal mucosa in a pre-selected sequence. Non-active containing layers in between these layers are useful to adjust timing of absorption/delivery for activation by UV, which is typically controlled by the thickness of such layers.

An example of administration through a mucosa in the oral cavity of a user is presented below. To treat a person with androgenetic alopecia, a mucosal patch according to any of the embodiments herein is placed on the soft palate or on the dorsal surface of tongue for the user to place the patch against the palate, which is then sucked on by the user. The patch will dissolve and will release the collagen carbon dot nanocomposite carrying the active agent to the palatal mucosa where it is absorbed into the blood stream for transport to the dermal papilla. After a pre-determined time period for delivery of a first active agent to the dermal papilla, typically 5-10 minutes, the target site is scanned with a deep blue LED of 210 nm for a determination of a sufficient concentration of the active agent at the target site. The scan may be over a range of wavelength from 210 to 510 nm, with the inclusion of some specific wavelength 210 nm, 260 nm, 310 nm, 400 nm, and 510 nm). The scan device can include a display or be in operative communication with an electronic device to provide a readout or other indication of the concentration of the active agent at the target site. The mucosal patches are configured to create a stable plasma level occurs within 2-4 minutes for each active agent. This is the time for the highest level of the active agent to be present in the plasma after a single dose administration.

Once a sufficient concentration is determined to be present, the target site is treated with a prescribed wavelength of radiation for a pre-selected treatment period of time, typically 2 minutes to 10 minutes, more preferably 5 minutes to 10 minutes. The wavelength is typically in a range of 260 to 510 nm but may be different for different active agents. For example, for finasteride 310 nm is used because this wavelength will separate the nanocomposite back into collagen, carbon dots or carbon nanotubes, and finasteride, which then activates the finasteride. A five-minute treatment with the 310 nm radiation was administered. Next, a deep blue LED 210 nm scan is again performed to confirm that the carbon dots or carbon nanotubes have disintegrated. This is another example of theragnostics.

Optionally, after waiting a secondary period of time for blood stream circulation delivery of additional drug nanoparticles to the target site, the deep blue scan and subsequent treatments by activating radiation are repeated. This portion of the treatment method is more likely with active agents of larger molecular weight because they take longer circulate out of the body.

In one embodiment, finasteride or finasteride and dutasteride are delivered first, and activated as described above, then flutamide is delivered second. UV rays are used to activate flutamide. The above process is repeated: administration, deep blue LED scan, treatment, and optionally a secondary time period with a secondary deep blue LED scan, and a second treatment of the same target site. Depending upon how much active agent is still in circulation after an hour or so, the secondary scan and secondary activation can be performed if enough of the nanocomposite is detected as the target site. The sequence of these active agents results in a reduction of dihydrotestosterone (DHT) in the dermal papilla by 70-90% (DHT causes hair loss) followed by blocking of the androgen receptors, thereby preventing any circulating natural DHT from attaching to the DHT receptor. The above dual active agent treatment can be repeated until a deep blue LED scan indicates a desired level of DHT. This creates a safe stable isolated compartment inside the dermal layer and isolates the dermal papilla from negative chemical exposure. All the destructive influence of 5AR2 on testosterone conversion into DHT and activation of androgen receptor(s) by DHT are removed resulting in a dormant DHT-AR influence.

Once the desired level of DHT is achieved, the above process is repeated with minoxidil as the active agent. Minoxidil will increase blood flow to the hair follicle bulb and also upregulate the ATPase via Ca²⁺ channel activation, thus accelerating the differentiation of the hair follicle stem cell and activating the growth of hair and/or new hair follicle growth. In one embodiment, the minoxidil is present in combination with the 5-Alpha Reductase 2-Inhibitor as the active agent in the drug nanoparticles.

Following the minoxidil treatment, the process is repeated with prednisone as the active agent. Prednisone in the dermal papilla activates P-450 dependent Aromatase (AROM). A high level of AROM may limit the formation of DHT (activity of 5α-R2 enzyme) which have higher affinity for the androgen receptor to initiate androgen cellular events. Thus, AROM may lower or down-regulate the DHT conversion from testosterone in hair follicles. This further magnifies the effect of provided by the sequential treatment with finasteride and flutamide.

The cleavage of the CL/CD nanoparticle also releases collagen in the dermal layer and dermal papilla. The radiation source may be an aluminum nitride LED having a wavelength of 210 nm. This is deep UV. The radiation source may be an excimer laser and/or mercury lamp having wavelengths in a range of 200-300 nm and a GaN LASER having a wavelength in a range of 300-400 nm. Existing scalp caps using LEDs for emitting wavelengths can be used if the cleavage wavelength is emitted.

In another embodiment, the above approach can be accomplished using a mucosal patch configured for the skin, such as the scalp.

### Use of Patch for Anti-Aging or Anti-Wrinkle Treatment

Delivery of an active agent to the skin is discussed below that treats wrinkles. The delivery is through a mucosal patch impregnated with liquified, dispersed, or consolidated dose(s) of a collagen carbon dot nanocomposite carrying an active agent, referred to as a drug nanoparticle. Example active agents include encapsulated insulin like growth factor (GF) or recombinant Human Growth Factor (rHGF). The collagen for these embodiments is typically of type I, II, III, IV, or V. When the skin to be treated is the face, the mucosal patch can be in the shape of a face mask. The mucosal patch can include vitamin E, A, C, B complex or combinations thereof and epidermal exfoliation chemicals. If the exfoliation chemicals are present in the mucosal patch, the patch is configured for the exfoliation to occur first. Known exfoliative chemicals are suitable for this application.

Here, activation using 210 nm wavelength releases activated collagen. This can include isolating properties of the collagen upon contact with skin at body temperature or warming or cooling system to certain specific temperatures. The collagen release occurs at the sub-dermal level where the collagen will be incorporated into the matrix of collagen providing additional strength and elasticity. The thickening and smoothening of the sub-dermal connective tissue layer flattens any wrinkles present.

This mucosal patch can also be used for treatment of skin after or during weight loss to prevent wrinkle formation and during weight gain as in weightlifting and bodybuilding, etc. to prevent striae. It may be used after or during pregnancy for reducing or removing skin wrinkles and reversing loss of elasticity that may have occurred with stretch marks.

Other applications of CL+CD+D might be present in specific collagen diseases like Ehlers-Danlos Syndrome, Chondrodysplasia, Osteogenesis Imperfecta and Alport's disease.

### Advantages

The palatal veins drain the palatal mucosa into the pterygoid venous plexus, which partially drains into the cavernous venous sinus through emissary veins and allows direct blood flow into the brain. The palatine venous plexus drains into maxillary vein and then to the external jugular system that takes blood back to the heart, thereby bypassing hepatic metabolism. Medications used in treatment of neurological or other diseases absorbed directly into the palatal mucosa to transport the medication into the blood stream bypasses hepatic first pass metabolism and bypasses the traditional arterial circulatory system. Thus, the mucosal patches greatly reduce the dose of the medication delivered to the human body, limits adverse effects, and limits complications of excessive dose or drug-to-drug interaction. For example, when administered to the palate of the oral cavity the active agent is delivered to the brain through the cavernous sinus, thus creating rapid onset of the action provided by the active agent and creates a steady state concentration in the brain.

The mucosal patch disclosed herein grants freedom to place the patch before going to bed to produce an immediate effect or a delayed effect without requiring the user to remember or awaken to take a dose of medication.

The nanocomposites (CL+CD+D) deliver a specific active agent (D) to a target site in inactive form. Likewise, the nanocomposite circulates in inactive form because the active agent is bound to the nanoparticle. Importantly, the active agent has no effect on other organs in the body. The active agent is activated by cleaving it from the collagen and carbon dot. This activation is selectively performed once the nanocomposite has reached a desired target site. Any remaining nanocomposites in circulation within the body are excreted from the body. If it remains in circulation and ultimately reaches the target cite it can be selectively active during an optional second activation cycle.

The nanocomposites (CL+CD+D) minimize side effects of each of the active agents because it does not activate in any other organ, for example problems of GI complications and erratic absorption therein are avoided. The active agent at delivery of the mucosal patch is inactive because it is bound to the collagen carbon dots. Hence, the active agent has no effect on other organs in the body. The activation of the active agent only occurs after being cleaved from this bound state. Any remaining drug (still attached to the nanoparticles) is excreted in urine or feces, or it may remain at the target site and can be activated in a second cycle upon completion of the first activation cycle if desired and purposefully activated.

It should be noted that the embodiments are not limited in their application or use to the details of construction and arrangement of parts and steps illustrated in the drawings and description. Features of the illustrative embodiments, constructions, and variants may be implemented or incorporated in other embodiments, constructions, variants, and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

Having described the invention in detail and by reference to preferred embodiments thereof, it will be apparent that modifications and variations are possible without departing from the scope of the invention which is defined in the appended claims.

1. A mucosal patch comprising:
a support layer supporting an active layer;
wherein the active layer comprises a collagen carbon dot nanocomposite carrying an active agent, and the collagen carbon dot nanocomposite is absorbable through a mucosa.

2. The mucosal patch of claim 1, wherein the support layer comprises a mixture of polytetrafluoroethylene (PTFE) selected from the group consisting of expanded-PTFE (e-PTFE), dense-PTFE (d-PTFE), and highly dense-PTFE (n-PTFE).

3. The mucosal patch of claim 2, wherein the support layer is a mixture of e-PTFE and d-PTFE having a ratio of ePTFE:d-PTE in a range of 4:1 to 1:4.

4. The mucosal patch of claim 1, wherein the support layer is dissolvable or bioabsorbable.

5. The mucosal patch of claim 4, wherein the support layer comprises any one or more of cellulose, gelatin, and collagen.

6. The mucosal patch of claim 1, further comprising a protective layer in direct contact with a mucosa-contact surface of the active layer, wherein the protective layer is dissolvable or bioabsorbable or is removable.

7. The mucosal patch of claim 5, wherein the protective layer is dissolvable, which is activated by moisture.

8. The mucosal patch of claim 5, wherein the protective layer is dissolvable and is activated by heat.

9. The mucosal patch of claim 5, wherein the patch comprising an adhesive outermost portion defining a perimeter of the active layer.

10. The mucosal patch of claim 1, wherein the active layer comprises a bioadhesive configured for adhesion to a mucosa.

11. The mucosal patch of claim 1, comprising an intermediate layer between the support layer and the active layer.

12. The mucosal patch of claim 1, wherein the active agent is absorbable through a mucosa selected from the group consisting of palate, tongue, cheek, gums, nasal, skin, eyes, ears.

13. A method of treatment comprising:
providing a mucosal patch comprising:
a support layer supporting an active layer;
wherein the active layer comprises a collagen carbon dot nanocomposite carrying an active agent, and the collagen carbon dot nanocomposite is absorbable through a mucosa;
contacting a mucosa of a user with the active layer of the mucosal patch for a period of time sufficient to deliver the collagen carbon dot nanocomposite to a target site in need of treatment; and
subsequent to contacting the mucosa, exposing the target site to a wavelength of radiation that cleaves the active from the collagen carbon dot nanocomposite.

14. The method of claim 13, further comprising, before exposing the target site, scanning the target site to determine if a treatment concentration of the active agent is present at the target site.

15. The method of claim 13, wherein the mucosa is the skin, and the active agent is selected from the group consisting of encapsulate insulin, vitamin E, vitamin A, vitamin C, vitamin B complex, vitamin D3, and combinations thereof.

16. The method of claim 15, wherein exposing the target site to a wavelength of radiation includes a wavelength of radiation to cleave the collagen from the carbon dot.

17. The method of claim 13, wherein the treatment is for androgenetic alopecia, the mucosa is in the oral cavity of the user, and the active agent comprises finasteride and/or dutasteride.

18. The method of claim 17, wherein the active agent comprises minoxidil.

19. The method of claim 18, wherein the active agent comprises flutamide and/or prednisone.
